(19) United States
(12) Patent Application Publication
(10) Pub. No.: US 2012/0164056 A1
(43) Pub. Date: Jun. 28, 2012

(54) GENERATION OF STERILANT GASSES AND USES THEREOF

(76) Inventors: Louis C. Haddad, Mendota Heights, MN (US); William E. Foltz, Cottage Grove, CA (US); Greggory S. Bennett, Hudson, WI (US); Robert A. Asmus, Hudson, WI (US)

(21) Appl. No.: 13/381,219

(22) PCT Filed: Jun. 28, 2010
(86) PCT No.: PCT/US10/40152

§ 371 Date: Mar. 6, 2012

Related U.S. Application Data
(60) Provisional application No. 61/222,376, filed on Jul. 1, 2009.

(51) Int. Cl. A61L 2/20; C09K 3/00; C01B 21/36

(52) U.S. Cl. 423/400; 252/182.11; 252/182.34; 252/182.33; 422/164; 422/149

(57) ABSTRACT

The disclosure provides processes and systems for sterilizing an object using a sterilant gas. In some embodiments, the sterilant gas is produced by the thermal decomposition of a salt. Compositions to generate sterilant gasses are also disclosed.

### CROSS REFERENCE TO RELATED APPLICATION

This application claims priority to U.S. Provisional Patent Application No. 61/222,376, filed Jul. 1, 2009, which is incorporated herein by reference in its entirety.

### BACKGROUND

Typical industry practices employ the use of moist heat (steam) or sterilant gasses (e.g., chlorine dioxide, hydrogen peroxide, nitric oxide, nitrogen dioxide, ozone, and ethylene oxide) to sterilize medical instruments or devices. In addition to logistical problems (e.g., bulky equipment and providing a source of steam), autoclaving is not suitable for many plastics and other heat labile materials.

Sterilant gases can kill or control the growth of microbial contaminations. One problem with many of the sterilant gases is that they typically can be used only in limited concentrations and they require special handling.

Certain sterilants, such as chlorine dioxide, ozone and hydrogen peroxide are difficult and expensive to transport. Many of these sterilant gases are powerful oxidizers. Gases, such as ozone and chlorine dioxide, must be generated at or near the point of use. On-site plants for generating one such sterilant gas, chlorine dioxide, are costly and require significant space to implement.

U.S. Pat. No. 6,607,696 describes a device for delivering chlorine dioxide to disinfect or sterilize a liquid or an item contained in the liquid. The device uses a permeable sachet containing gas generating reactants, such as sodium chlorite and citric acid, where the sachet is a receptacle permeable to liquid and gas. Liquid can diffuse into the receptacle to reach the gas generating reactants that then generate a gas, such as chlorine dioxide. The gas that diffuses out of the permeable sachet is not sealed from the environment/atmosphere. Chlorine dioxide can be produced in multi-compartmental devices that employ gas-generating ingredients contained in liquid- and gas-permeable compartments, such as the multi-compartment devices described in U.S. Pat. Nos. 6,602,466 and 6,607,696. Not only are these systems expensive and difficult to manufacture, but they do not provide predictable/controllable release of the gas into the sterilizing chamber and they may not prevent the unintended escape of sterilant gas to the environment.

Thus, there is a need for simple, safe, inexpensive methods and devices that generate sterilant gases at the point of use in a safe and efficient manner.

### SUMMARY

The present disclosure generally provides processes to generate and use one or more sterilant gasses from inorganic salts. The present disclosure further provides a process to generate the sterilant gas in situ in a sterilizer. In some embodiments, one or more sterilant gas is generated by thermal decomposition of a thermolabile salt. In some embodiments, one or more sterilant gas is generated by a redox reaction including a metal and an acid.

In some embodiments, the sterilant gasses include oxides of nitrogen that can be used for the purpose of sterilization, decontamination, and/or disinfecting. In some embodiments, the sterilant gasses include oxides of chlorine that can be used for the purpose of sterilization, decontamination, and/or disinfecting. The oxides of nitrogen may include, for example, nitric oxide, nitrogen dioxide, dinitrogen tetroxide or additional oxides of nitrogen individually or in combination. In addition, the mixture of nitrogen oxide gases generated in methods of the present disclosure has lower oxidation potential than other sterilant gases. The oxides of chlorine may include, for example chlorine dioxide.

Thus, in one aspect, the present disclosure provides a process of producing a sterilant gas. The process can comprise providing a source of thermal energy and a mixture comprising a desiccant and a thermolabile salt. The process further can comprise heating the mixture to a temperature sufficient to cause decomposition of the salt to a nitrogen oxide.

In some embodiments, the nitrate salt can comprise Ba(NO₃)₂, AgNO₃, Fe(NO₃)₃.9H₂O, Cu(NO₃)₂.2.5H₂O, Ca(NO₃)₂.4H₂O, Mn(NO₃)₂.4H₂O, Co(NO₃)₂.6H₂O, or Zn(NO₃)₂.xH₂O. In any of the above embodiments, heating the thermolabile salt can comprise heating the salt in the presence of oxygen. In any of the above embodiments, the salt can be disposed in a package adapted for heating the salt to a temperature sufficient to cause decomposition of the salt to a sterilant gas. In some embodiments, the sterilant gas can be an oxide of nitrogen.

In another aspect, the present disclosure provides a process for sterilizing an object. The process can comprise contacting the object in a sterilizer with a sterilant gas generated by thermal decomposition of a thermolabile salt. In some embodiments, the process further can comprise providing an object to be sterilized, a sterilizer, a source of thermal energy, and thermolabile salt. The salt can be capable of decomposing at an elevated temperature to generate a sterilant gas. The process further can comprise placing the object in the sterilizer. The process further can comprise heating the thermolabile salt to generate an amount of sterilant gas effective to cause sterilization of the object, wherein the sterilant gas is received in the sterilizer. The process further can comprise contacting the object with the sterilant gas in the sterilizer for a period of time. In some embodiments, the process further can comprise heating the salt in the sterilizer. In some embodiments, heating the thermolabile salt can comprise heating the thermolabile salt in a gas-generating chamber that is in selective fluid communication with the sterilizer.

In any of the above processes, the process further can comprise exposing the object to be sterilized to humidified air before, during, and/or after contacting the object with the sterilant gas. In any of the above processes, exposing the object to humidified air can comprise exposing the object to relative humidity in the range from about 30 percent to about 99 percent. In any of the above processes, heating the thermolabile salt can comprise heating the salt in a package adapted for heating the salt to a temperature sufficient to cause decomposition of the salt. In any of the above processes, heating the thermolabile salt can comprise heating the salt in the presence of oxygen. In any of the above processes, heating the thermolabile salt in a package can comprise heating an amount of thermolabile salt in the package sufficient for a single sterilization process. In any of the above embodiments, heating the thermolabile salt can comprise heating a thermolabile salt admixed with a desiccant.

In another aspect, the present disclosure provides a composition for generating a sterilizing gas. The composition can comprise a desiccant and a thermolabile salt. The thermolabile salt can be capable of decomposing at an elevated temperature to generate a sterilant gas. On a mass basis, the composition can comprise greater than one part thermolabile salt hydrate per nine parts desiccant.

In another aspect, the present disclosure provides a system for sterilizing an object. The system can comprise a sterilization chamber, a heat source, and a thermolabile salt. The thermolabile salt can be capable of decomposing at an elevated temperature to generate a sterilant gas. In some embodiments, the sterilization chamber is sealable. In any of the above embodiments, the system can further comprise a gas-generating chamber. In any of the above embodiments, the system can further comprise a source of oxygen. In some embodiments, the sterilization chamber can be in fluid connectivity with the source of oxygen. In any of the above embodiments, the system can further comprise a source of moisture vapor. In some embodiments, the sterilization chamber can be in fluid connectivity with the source of moisture vapor. In any of the above systems, the thermolabile salt can be disposed in a package adapted for heating the salt to a temperature sufficient to cause decomposition of the salt. In any of the above embodiments, the system can further comprise a gas-scrubbing component.

In any of the above processes, compositions, or systems, the thermolabile salt can comprise an inorganic salt. In any of the above processes, compositions, or systems, the inorganic salt can comprise a nitrate salt, a nitrite salt, a chlorate salt, a perchlorate salt, or mixtures thereof. In any of the above processes, compositions, or systems, the thermolabile salt can comprise a salt hydrate. In any of the above processes, or systems providing a thermolabile salt can comprise providing a predetermined amount sufficient to attain an effective amount of sterilant gas in the sterilizer. In any of the above processes, or systems, heating the thermolabile salt can comprise heating the salt to at least about 100 centigrade. In any of the above processes, or systems, heating the thermolabile salt can comprise heating the salt in the presence of oxygen. In any of the above processes, compositions, or systems, the thermolabile salt can be admixed with a desiccant. In any of the above embodiments, the desiccant can comprise a molecular sieve, clay, anhydrous potassium sulfate, anhydrous calcium sulfate, an inorganic oxide, or mixtures thereof. In some embodiments, the inorganic oxide can be selected from, for example, silicon dioxide, aluminum oxide, zirconium oxide, or mixtures thereof.

In another aspect, the present disclosure provides a process for sterilizing an object. The process can comprise contacting the object in a sterilizer with a sterilant gas generated by the reaction of an oxidizable metal with an acid. In some embodiments, the process further can comprise providing an object to be sterilized, a sterilizer, an oxidizable metal, and an acid. The acid can be reduced to generate a sterilant gas. The process further can comprise placing the object in the sterilizer and contacting the oxidizable metal with the acid to generate an effective amount of sterilant gas. The sterilant gas can be received in the sterilizer. The process further can comprise contacting the object with the sterilant gas in the sterilizer for a period of time. In some embodiments, the acid can comprise nitric acid. In some embodiments, the oxidizable metal can comprise copper.

In another aspect, the present disclosure provides a system for sterilizing an object. The system can comprise a sterilization chamber, an oxidizable metal, and an acid that can be reduced to generate a sterilant gas.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, “an” object to be sterilized can be interpreted to mean “one or more” objects to be sterilized.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawing figures listed below, where like structure is referenced by like numerals throughout the several views.

FIG. 1 is a cross-sectional view of one embodiment of a sterilization system according to the present disclosure.

FIG. 2 is a side view, partially in cross-section, of another embodiment of a sterilization system according to the present disclosure.

FIG. 3A is cross-sectional view of a cartridge containing a thermolabile salt.

FIG. 3B is top view of the cartridge of FIG. 3A.

### DETAILED DESCRIPTION

Before any embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways. Also, it is to be understood that the phraseology and terminology used herein is for the purpose of description and should not be regarded as limiting. The use of “including,” “comprising,” “containing,” or “having” and variations thereof herein is meant to encompass the items listed thereafter and equivalents thereof as well as additional items. Unless specified or limited otherwise, the terms “supported,” and “coupled” and variations thereof are used broadly and encompass both direct and indirect supports and couplings. It is to be understood that other embodiments may be utilized and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

The present disclosure is generally directed to methods and articles for generating and using sterilant gasses to disinfect or sterilize objects. In certain preferred embodiments, the present disclosure provides methods and devices that generate or use nitrogen dioxide, along with other oxides of nitrogen, to sterilize or disinfect instruments, devices, materials, tools and equipment that must be sterile, typically for medical applications. The use of nitrogen dioxide alone, or in combination with oxides of nitrogen that form in combination with air, as a disinfectant and sterilant gas mixture has several advantages over other gases. Neither nitrogen dioxide nor other oxides of nitrogen are combustible at high concentrations. In addition, because nitrogen dioxide and other oxides of nitrogen have weaker oxidizing potential than peroxides and ozone, they allow for a broader list of materials that can be sterilized.

Generating a mixture of the oxides of nitrogen can have additional advantages over pure nitric oxide and other single entity sterilization gases. Nitric oxide is very lipid soluble and has the ability to disrupt the lipid membranes of microorganisms. Furthermore nitric oxide may inactivate thioproteins thereby disrupting the functional proteins of microbes. Nitrogen dioxide is more water soluble than nitric oxide. Finally, nitric oxide and nitrogen dioxide are extremely effective disruptors of DNA, causing strand breaks and other damage leading to an inability for the cell to function.

A mixture of nitric oxide and air will react, resulting in a mixture containing many different oxides of nitrogen. Specifically, the addition of NO to air, or air to NO, results in the formation of NO₂, when NO reacts with the oxygen in air. The concentration of each nitrogen-oxide species that is present in a mixture will vary with temperature, pressure, and initial concentration of the nitric oxide.

### Definitions

As used herein, the term “gas” or “gases” means any matter that is not in the solid state or liquid state, but rather, has relatively low density and viscosity, expands and contracts greatly with changes in pressure and temperature, diffuses readily and has the tendency to become distributed uniformly throughout any container.

As used herein, the term “nitric oxide” or “NO” means the NO free radical or NOₓ. As used herein, the term NOₓ is an abbreviation for nitrogen oxides or the oxides of nitrogen, which are the oxides formed by nitrogen in which nitrogen exhibits each of its positive oxidation numbers from +1 to +5. As used herein, the terms “nitrogen oxides” and ‘oxides of nitrogen’ and ‘NO,’ mean a gas having one or more of the following gases, all of which contain nitrogen and oxygen in varying amounts: nitric oxide (NO), nitrogen dioxide (NO₂), dinitrogen trioxide (N₂O₃), dinitrogen tetroxide (N₂O₄), dinitrogen pentoxide (N₂O₅) and nitrous oxide (N₂O). Examples of preferred sterilant gases include, but are not limited to NO, NO₂, N₂O₃, N₂O₄, N₂O₅, N₂O and mixtures thereof. Examples of the most preferred sterilant gases are NO, NO₂, N₂O₄ and mixtures thereof.

As used herein, the term “NOₓ-generating” compound or composition means a compound or composition capable of producing or releasing NO, NO₂, and NOₓ. As used herein, the term “sterilant gas-generating” compound or composition means a compound or composition capable of producing or releasing a sterilant gas. An NOₓ-generating compound is one type of sterilant gas-generating compound. The preferred NOₓ-generating compounds used in the systems, devices and methods of the present invention are inorganic salt compounds. More preferred NOₓ-generating compounds include those that generate at least 1 mole of NO₂ per mole of compound. Even more preferred NOₓ-generating compounds include those that generate at least 2 moles of NO₂ per mole of compound. Even more preferred NOₓ-generating compounds include those that generate at least 3 moles of NO₂ per mole of compound.

As used herein, the term “sterilization chamber” means any sealed chamber of any size in which items to be sterilized, disinfected, or decontaminated can be contained. Preferably, the sterilization chamber is capable of maintaining a vacuum; receiving a sterilizing gas; and receiving air. Sterilization is a high-level of decontamination that destroys all microbial life, including highly resistant bacterial endospores. Disinfection is an intermediate-level of decontamination, which eliminates virtually all pathogenic microorganisms, with the exception of bacterial spores. As used herein, the terms “sterilize”, “sterilizing” and “sterilization” mean the killing or removal of all microorganisms in a material or on an object. When a material or object is “sterilized” or “sterile” there are no living organisms in or on a material or object. Since sterilization eliminates all microorganisms, including endospores, a method, system and/or device that sterilizes a material or object, therefore, also disinfects and decontaminates the material or object.

As used herein, the term “object” refers not to a feature of the invention, but rather to the article or material being acted upon to be sterilized, disinfected, and/or decontaminated by the disclosed sterilizing methods, systems and devices. The term “object” can also include a material to be sterilized, no matter the physical form. An object may include, for example, without limitation, a medical device or medical instrument or any other article or combination of articles for which sterilization is desired. An object may have a wide variety of shapes and sizes and may be made from a variety of materials (e.g., without limitation, metal, plastic, glass).

As used herein, the term “gas generation chamber” means any container, of any size or composition, which may be used to contain a gas and/or a gas-generating compound. Preferably, the gas generating chamber is made of a material that is impermeable to liquid and impermeable to gas.

As used herein, the term “microbe” means any bacteria, virus, fungi, yeast, parasite, mycobacterium or the like.

As used herein, the term “scrubbing” means the removal or conversion of toxic gasses (e.g., oxides of nitrogen) from the exhaust stream of the sterilization device.

As used herein, the term “medical device” means any instrument, apparatus, implement, machine, appliance, contrivance, implant, or other similar or related article, including any component, part, which is intended for use in the cure, mitigation, treatment, or prevention of disease, of a human or animal, or intended to affect the structure or any function of the body of a human or animal; and, which is intended to be inserted, in whole or in part, into intact tissues of a human or animal. As used herein, the term “implant” or “implantable” means any material or object inserted or grafted into intact tissues of a mammal.

As used herein, the term “impermeable” means a substance, material or object that prohibits over 95% of any liquid or gas from passing or diffusing through it, for at least one hour. As used herein, the term “permeable” means a substance, material or object that allows the passage of gases and/or liquid through it.

The sterilization system and method of the present disclosure utilizes one or more inorganic salts, in thermal contact with a heat source, to generate a sterilant gas. The sterilant gas is contacted with an object, preferably in a sealed chamber, for a predetermined length of time to effect the sterilization of the object.

Sterilization systems and methods of the present disclosure employ compounds that release a sterilant gas, preferably nitrogen dioxide, upon heating. The systems and methods of the present disclosure generate nitrogen oxides that may be used as a mixture of water soluble and lipid soluble nitrogen oxide gases, to sterilize a wide variety of devices, instruments, materials, human and animal tissues, drugs, biologicals, and a variety of medically relevant materials. In one embodiment of the present invention, the object to be sterilized is made of a material that is used in medical devices. Examples of medical devices are, without limitation, all types of surgical instruments; cardiac surgery products; cardiac implants; cardiovascular stents; vascular implants; orthopedic surgery products such as surgical instruments, bone graft, bone scaffold; orthopedic implants; dental surgery products; dental implants; gastrointestinal implants, urinary tract implants; wound healing products; tissue engineering products. In another embodiment of the present invention, the tissue engineering product is a protein.

Typically, an object that is a medical device contains one or more materials such as, for example, metals, non-metals, polymers or plastics, elastomers, and/or biologically derived materials. Preferred metals used in medical devices are stainless steel, aluminum, nitinol, cobalt chrome, and titanium. Non-limiting examples of nonmetals are glass, silica, and ceramic.

In another embodiment of the present invention, the object to be sterilized is made of a material that is a polymer such as a polyester bioresorbable polymer, for example, without limitation, Poly(L-lactide), Poly(DL-Lactide), 50/50 Poly(DL-lactide-co-glycolide), Poly(e-caprolactone), and mixtures thereof. Preferably, the material is a bioresorbable polymer capable of being used as an implant material and for drug delivery. Preferred polymers used in medical devices are polyacetal, polyurethane, polyester, polytetrafluoroethylene, polyethylene, polymethylmethacrylate, polyhydroxyethyl methacrylate, polyvinyl alcohol, polypropylene, polymethylpentene, polyetherketone, polyphenylene oxide, polyvinyl chloride, polycarbonate, polysulfone, acrylonitrile-butadiene-styrene, polyetherimide, polyvinylidene fluoride, and copolymers and combinations thereof. Other materials found in medical devices are polysiloxane, fluorinated polysiloxane, ethylenepropylene rubber, fluoroelastomer and combinations thereof. Examples of biologically derived materials used in medical devices include, without limitation, polylactic acid, polyglycolic acid, polycaprolactone, polyparadioxanone, polytrimethylene carbonate and their copolymers, collagen, elastin, chitin, coral, hyaluronic acid, bone and combinations thereof.

Certain types of medical devices and implants include a bioactive coating and/or biocompatible coating, examples of which are, without limitation, infection resistance coating, antimicrobial coating, drug release coating, antithrombogenic coating, lubricious coating, heparin coating, phophoryl choline coating, urokinase coating, rapamycin coating, and combinations thereof. The bioactive coating can be a hydrophilic or hydrophobic coating. Further examples of bioactive coatings and polymers include, but are not limited to polyvinyl pyrrolidone, polyethylene glycol, polypropylene glycol, polyethylene glycol-co-propylene glycol, polyethylene glycol acrylate, polyethylene glycol diacrylate, polyethylene glycol methacrylate, polyethylene glycol dimethacrylate, polyethylene oxide, polyvinyl alcohol, polyvinyl alcohol-co-vinylacetate, polyhydroxyethyl methacrylate, and polyhyaluronic acid, and hydrophilically substituted derivatives, monomers, unsaturated pre-polymers, and uncrosslinked polymers with double bonds thereof. Addition bioactive coatings and polymers are polytetrafluoroethylene, polyethylene, polypropylene, poly-(ethylene terephthalate), polyester, polyamides, polyarylates, polycarbonate, polystyrene, polysulfone, polyethers, polyacrylates, polymethacrylates, poly(2-hydroxyethyl methacrylate), polyurethanes, poly(siloxane)s, silicones, poly(vinyl chloride), fluorinated elastomers, synthetic rubbers, poly(phenylene oxide), polyetherketones, acrylonitrile-butadiene-styrene rubbers, poyetherimides, and hydrophobically substituted derivatives thereof and their precursor monomers.

In another embodiment of the present disclosure, the object to be sterilized is made of a material that is a bioabsorbable polymer or a drug-bearing or a drug-eluting polymer or mixtures thereof. In a preferred embodiment of the present disclosure, the object to be sterilized is an implant.

### Nitrogen Oxides:

In some embodiments, the sterilization system and method of the present disclosure utilizes one or more oxides of nitrogen (individually or in combination) to sterilize a wide variety of devices, instruments, materials, human and animal tissues, drugs, biologicals, and a variety of medically relevant materials.

Oxides of nitrogen can be generated by heating nitrite or nitrate salts to a temperature sufficient to decompose the salt. Exemplary reactions of thermal decomposition of nitrite and nitrate salts are shown in the following formulae:

4Fe(NO₃)₃.9H₂O→36H₂O+2Fe₂O₃+12NO₂+30₂ (1)

2Cu(NO₃)₂.2.5H₂O→5H₂O+CuO+4NO₂+O₂ (2)

where the metal cation (Fe⁻³ and Cu⁺², respectively, in these equations), can be, for example, any metal cation selected from Periodic Table Group IIA or Group IIIA elements. For example, the metal could be selected from the group consisting of Mg, Ca, Ag, Ni, Sr, Ba, Mn, Fe, Co, Cu, Pb, Ga, Bi, and Zn. Preferred embodiments include nitrate salts that produce a relatively high yield of nitrogen dioxide at a relatively low temperature. Preferred embodiments also include nitrate salts that decompose to, in addition to nitric oxide, relatively safe, stable products. A particularly preferred embodiment includes the thermal generation of nitrogen dioxide from ferric (III) nitrate hydrate, which decomposes to nitrogen dioxide and ferric oxide (rust) at a relatively low temperature (about 117 degrees centigrade).

Nitric oxide (NO) generated from the decomposition reaction can react with oxygen to form nitrogen dioxide, as shown in the following formula:

2NO+O₂→2NO₂ (3)

The oxygen used to convert nitric oxide to nitrogen dioxide may be provided by thermal decomposition of the salt. In some embodiments, the oxygen used to convert nitric oxide to nitrogen dioxide may be provided by air. In some embodiments, the oxygen used to convert nitric oxide to nitrogen dioxide may be provided by substantially pure oxygen or by a mixture of gasses comprising oxygen.

A preferred embodiment of the system and method of the present disclosure generates the gases at the point-of use. Such point-of-use methods, systems and devices eliminate the need for heavy tanks of gases or expensive on-site gas generation plants. In one aspect, the present disclosure describes a method to generate a mixture of nitrogen oxides for sterilization and disinfecting purposes. In some embodiments, method employs an apparatus that integrates the gas generation and delivery method. The apparatus used in the process may have many potential embodiments.

In a preferred embodiment of the system or device of the present disclosure, a sterilization chamber is used, along with a source of the sterilant gas comprised of one or more oxides of nitrogen. The sterilization chamber may be in fluid connectivity with the source of the sterilant gas; alternatively, the source of the sterilant gas can be within the sterilization chamber. One preferred embodiment includes a gas generation chamber in fluid connectivity with a sterilization chamber. Another preferred embodiment has the gas generation chamber contained within the sterilization chamber.

Also preferred are embodiments of the system and method of the present disclosure that produce a mixture of nitrogen oxides having less oxidative potential than commonly used sterilant gases, including ozone and hydrogen peroxide. An additional advantage is that the mixture of nitrogen oxides produced is noncombustible. This allows the use of high concentrations of the gaseous mixture the system and method of the present invention thereby allowing short exposure times in the sterilization cycles than are used with other sterilant gasses.

Yet another advantage of the method of the present disclosure is that multiple chemical species with different chemical properties are generated for the purpose of sterilization and disinfecting. Those skilled in the art understand that multiple mechanisms of cell killing or deactivation are often preferred over single mechanisms of action. Antimicrobial agents with different mechanisms of action are often synergistic when used together, producing a greater effect than would be expected by simply adding the effects from each agent together.

In one preferred embodiment of the method and system of the present invention, NO₂ gas is generated using the class of NOₓ-generating compounds known as nitrite or nitrate salts. These compounds spontaneously release NO₂ upon heating to a temperature sufficient to decompose the compound. Elevated temperatures can be used to generate NO₂ rapidly in the method of the present disclosure.

The NOₓ-generating compounds utilized in the systems and methods of the present invention provide several advantageous elements. Nitrogen dioxide, and other oxides of nitrogen such as dinitrogen tetroxide, are more water soluble than nitric oxide. These, and especially nitrogen dioxide, are highly damaging to DNA, resulting in nitrosation and deamination of DNA bases and single and double strand breaks. Damage to DNA is a powerful killing mechanism. The mixture of gases in the present disclosure provides a multipronged attack of microbes through a variety of possible mechanisms of action.

Another embodiment of the system and method of the present disclosure uses a gas generating chamber that is a pressurized or non-pressurized cylinder containing one or more nitrogen oxide-generating compounds. The gas or gas mixture generated from the one or more nitrogen oxide-generating compounds can be delivered to the sterilization chamber through a valve or a metered regulator in fluid connectivity with the sterilization chamber, or other gas delivery method known to one skilled in the art. Another embodiment includes computer or microprocessor means to control the delivery of sterilant gas from the cylinder.

A preferred embodiment of the system and method of the present invention includes a gas generation chamber containing both a salt (e.g., an inorganic nitrate salt), whereby the gas generation chamber includes or is in thermal contact with a heat source that allows the gas generation chamber to be heated to a temperature sufficient to cause decomposition of the nitrogen dioxide-generating salt, and is in fluid connectivity with the sterilization chamber so that gas generated upon heating of the salt is transported into the sterilization chamber. Additional connections and/or ports may be included for such purposes as to introduce air and/or water vapor into the sterilization chamber. Additional connections may also include a vacuum source to evacuate air and/or nitrogen oxides from the sterilization chamber. Preferably, the NO₂ gas is released into a reusable NO₂, scrubbing system. Preferred methods and devices of the present disclosure include the scrubbing of the sterilant gas after the object is sterilized.

One skilled in the art can apply simple calculations to determine the number of moles of thermolabile salt needed generate NO₂ to achieve a desired concentration of NO₂ in the defined volume of a sterilization chamber. Because the effectiveness of a sterilization process is related to the concentration of the sterilant gas and the length of exposure time, this can allow the user to control the amount of NO₂ added for various sterilization applications. For example, medical practitioners may desire a more rapid sterilization cycle, requiring higher concentrations of added NO₂. Those users who are more concerned with portability may be less sensitive to speed and cost of the process. Longer sterilization cycles may require less of the NO₂-releasing compound, i.e., less NO₂ added. Thus, the devices and processes of the present disclosure offer the flexibility to provide potential end users with options regarding cost, speed, portability, and other utilization parameters.

The system and methods of the present disclosure preferably include a system that can remove and/or detoxify the sterilant gases, otherwise known as scrubbing. The method of the present disclosure preferably includes a scrubbing process that removes and detoxifies these gases, prior to retrieving the sterilized or disinfected materials from the sterilization chamber. The scrubbing process includes numerous methods for removing and/or reacting with the NO, NO₂, and NOₓ. Scrubbing systems and processes may employ an adsorbent to trap NO₂, and an oxidizer to convert NO to NO₂. In appropriate conditions, the sterilant gas may be exhausted to the outside environment, where the concentrations of NO, NO₂, and NOₓ, will dissipate easily. The scrubbing process may be achieved using a commercially available scrubbing device, such as the Buchi Analytical B-414 (New Castle, Del.). Preferably, the scrubbing device reduces the levels of NO, NO₂, and NOₓ, in the exhaust gas to levels that are safe and in accordance with local regulatory requirements. It is also preferred that the entire method, including a scrubbing process, can be performed in a short amount of time.

In a preferred embodiment, the gases are removed from the chamber prior to opening the chamber. In some instances such as outdoor use, the chamber may be opened without prior removal of gases.

### Sterilization Devices and Systems:

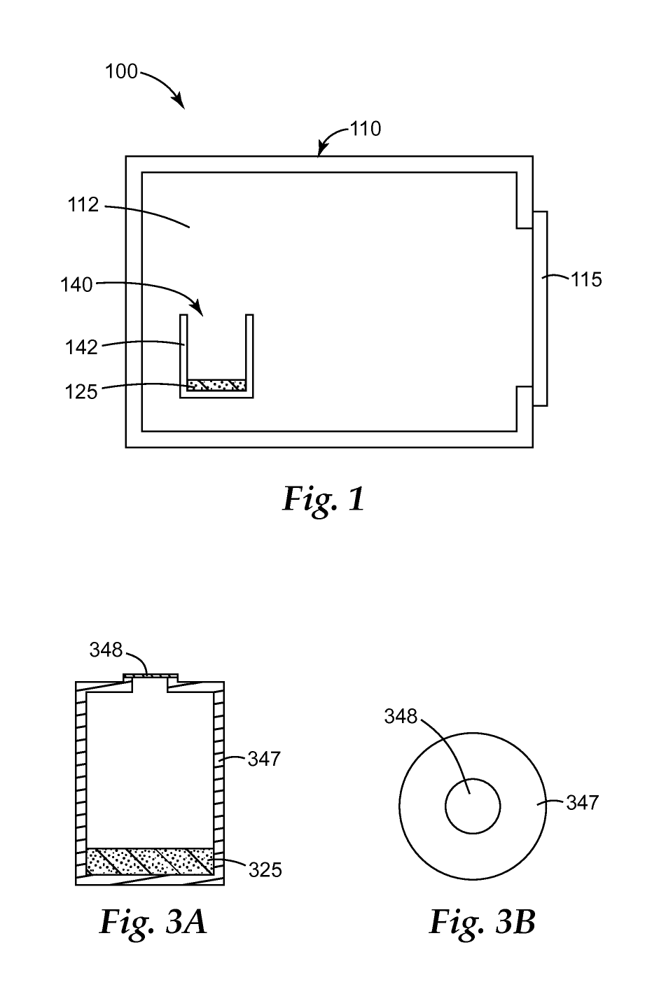

The present disclosure includes devices and systems for sterilizing an object. FIG. 1 shows one embodiment of a sterilizing system 100. The system 100 comprises a sterilizer 110 including a sealable chamber 112 with a closure 115. The sealable chamber 112 and closure 115 are preferably constructed of any suitable material (e.g., stainless steel) that is substantially impervious to gaseous sterilants such as oxides of nitrogen, for example. In certain preferred embodiments, the sealable chamber 112 and closure 115 are impervious to water vapor. The sterilizer 110 further comprises a gas-generating module 140. The gas-generating module 140 comprises a receptacle 142. Receptacle 142 receives thermolabile salt 125 or mixtures thereof. In some embodiments, the thermolabile salt may be disposed in a sachet.

Receptacle 142 may comprise a source of thermal energy (e.g., a heating coil, not shown) or, alternatively, may be thermally coupled to a source of thermal energy (not shown). The source of thermal energy should be capable of heating the thermolabile salt 125 to a temperature at which the salt decomposes to release a sterilant gas. The receptacle 142 can be constructed of materials suitable to withstand temperatures high enough to decompose the thermolabile salts or mixtures thereof. “Thermally coupled”, as used herein refers to a condition wherein thermal energy can be transmitted (e.g., by convection, conduction, or radiation) from the source of thermal energy to the receptacle 142 and/or the contents therein. In some embodiments, the source of thermal energy also may be used to elevate and/or control the temperature of the sealable chamber 112. In some embodiments, the receptacle 142 and/or the source of thermal energy may be insulated to minimize the transfer of heat to the sealable chamber 112 and/or objects therein. It should be noted that it is known in the art that certain thermolabile salts (e.g., metal nitrate salts) can comprise small amounts of corrosive acid (e.g., nitric acid). Therefore, in preferred embodiments, the receptacle is constructed from materials that are chemically resistant to the potential corrosive effects of the thermolabile salt and/or the products of thermal decomposition of the thermolabile salt.

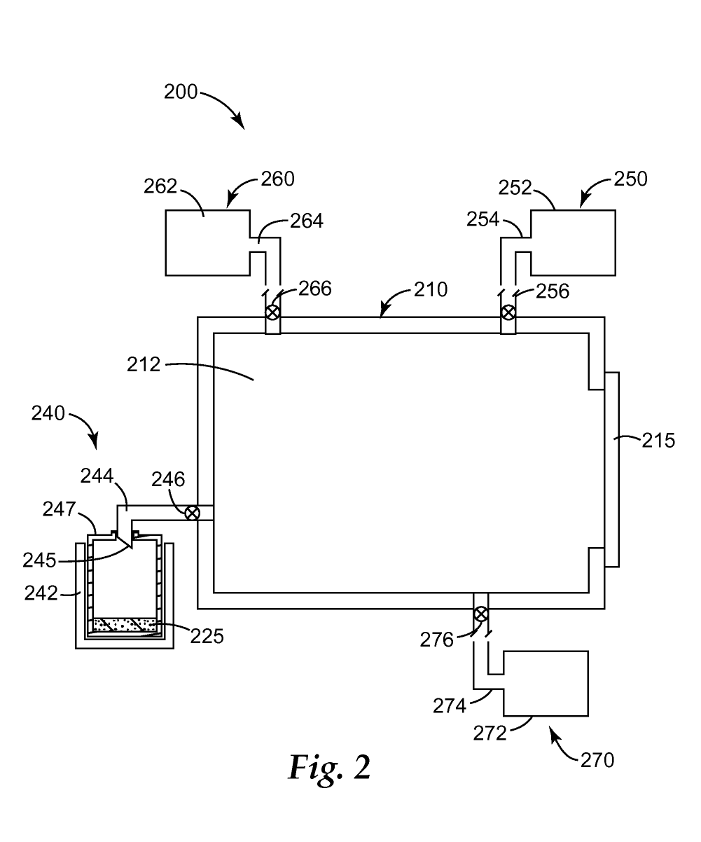

FIG. 2 shows another embodiment of a sterilization system 200 according to the present disclosure. The system 200 comprises a sterilizer 210 including a sealable chamber 212 with a closure 215, both as described above. The system further comprises a gas-generating module 240. The gas-generating module 240 comprises a receptacle 242 and a sterilant cartridge 247. Receptacle 242 may comprise a source of thermal energy (e.g., a heating coil, not shown) or, alternatively, may be thermally coupled to a source of thermal energy (not shown). The receptacle 242 and sterilant cartridge 245 can be constructed of materials suitable to withstand temperatures high enough to decompose the thermolabile salt 225 or mixtures thereof.

Sterilant cartridge 247 is in fluid communication with sealable chamber 212 through gas conduit 244. Gas conduit 244 can further comprise an optional gas conduit control valve 246, to regulate the flow of sterilant gas from the gas-generating module 240 to the sealable chamber 212. Gas conduit 244 may include a piercing member 245 to penetrate optional seal 248 on the cartridge 247. The gas conduit 244 is preferably constructed from materials that are substantially impervious to one or more of the sterilant gasses disclosed herein and all connections between the gas-generating module 240 and the sealable chamber 212 are preferably gas-tight. Suitable materials for the gas conduit 244 and gas conduit control valve 246 for sterilization processes involving oxides of nitrogen are described in U.S. Patent Application Publication No. US 2007/0014686 A1, which is incorporated herein by reference in its entirety.

In any of the above embodiments, the system 200 can further comprise an optional water vapor module 250 to provide and/or regulate the relative humidity in the sealable chamber 212. The moisture vapor module 250 can comprise a moisture vapor source 252 (e.g., a container of water, a vaporizer, a steam line); a moisture vapor conduit 254; and a moisture vapor control valve 256, which controls the fluid communication between the moisture vapor source 252 and the sealable chamber 212. Although the water vapor module 250 is shown external to the sealable chamber 212, it is recognized that the module 250 could be as simple as a receptacle of water, optionally coupled to a source of thermal energy, positioned in the sealable chamber 212 (not shown).

In any of the above embodiments, the system 200 can further comprise an optional compressed gas module 260. The compressed gas module 260 can advantageously provide gas flow into and/or out of the sealable chamber 212. Additionally, the compressed gas module can be used to maintain positive pressure within the sealable chamber 112 and/or it may be used to provide oxygen to the sealable chamber. The compressed gas module 260 can comprise a compressed gas source 262 (e.g., an air compressor, a compressed gas cylinder, a compressed oxygen cylinder), a compressed gas conduit 264, and a compressed gas control valve 266, which controls the fluid communication between the compressed gas source 262 and the sealable chamber 212. Compressed gas control valve 266 is preferably constructed from materials that are impervious to water vapor and the gaseous sterilants disclosed herein.

In any of the above embodiments, the system 200 can further comprise an optional vent module 270 for permitting gas flow out of and/or maintaining negative pressure within the sealable chamber 212. Vent module 270 may be as simple as a vent control valve 276 that controls the release of gaseous contents of the sealable chamber 212 to the external environment. Vent module can further comprise an optional vacuum source 272 (e.g., a vacuum pump) in fluid communication with the vent control valve 276 via the vent conduit 274. The vent module 270 may further comprise or may be operationally coupled to a gas-scrubbing component (not shown) that can remove a portion or all of the sterilant gas before it is evacuated from the sterilizer 210. Suitable scrubbers to remove oxides of nitrogen are described in U.S. Patent Application Publication No. US 2007/0014686 A1.

FIG. 3A shows a cross-sectional view of the sterilant cartridge of FIG. 2. The cartridge 347 can be formed from suitable materials (e.g., metals) that can tolerate the temperatures at which the thermolabile salts disclosed herein decompose. Preferably, the materials remain substantially impervious to gaseous sterilants over the complete range of operational temperatures. Also shown in FIG. 3A are a thermolabile salt 325 and an optional seal 348. The seal 348 functions to contain the thermolabile salt 325 in the sterilant cartridge 345 during shipping, storage, handling, and operational usage. In some embodiments, the seal 348 may be a friction-fit cap or a screw cap. In some embodiments, the seal 348 may be a frangible seal formed of paper, cardboard, polymeric film, metal (e.g., metal foil), or derivatives or combinations thereof. In use, preferably the cartridge 345 and/or the seal 348 form a gas-tight connection with the gas conduit (244, FIG. 2) or the like. FIG. 3B shows a top view of the sterilant cartridge 347 and optional seal 348 of FIG. 3A.

### Process for Generating a Sterilant Gas by Thermal Decomposition of a Salt:

The present disclosure provides processes for generating a sterilant gas. In some embodiments, the process comprises providing a compound comprising a thermolabile salt (e.g., a nitrate salt, a nitrite salt, a chlorate salt, perchlorate, or mixtures thereof) and a source of thermal energy. Preferred thermolabile salts include inorganic thermolabile salts. The method further comprises heating the thermolabile salt to a temperature sufficient to cause the decomposition of the salt to a sterilant gas.

The salt can comprise, for example, any metal cation selected from Periodic Table Group IIA or Group IIIA elements. For example, the metal cation could be selected from the group consisting of Mg, Ca, Ag, Ni, Sr, Ba, Mn, Fe, Co, Cu, Pb, Ga, Bi, and Zn. Preferred embodiments include salts that produce a relatively high yield of sterilant gas at a relatively low temperature.

Thermolabile salts of the present disclosure decompose to produce sterilant gasses that kill biological cells. The sterilant gasses include, for example, nitrogen dioxide, and chlorine dioxide.

In some embodiments, the salt can be a salt hydrate. In some embodiments, heating the salt hydrate can cause the salt to liquefy before or during the decomposition of the compound. As the temperature of the liquid mixture continues to rise, the liquid mixture may sputter, thereby potentially disrupting and/or delaying the decomposition process. In some embodiments, the thermolabile salt can be admixed with a desiccant. Preferably, the thermolabile salt is admixed with the desiccant. Even more preferably, the desiccant can be uniformly admixed (e.g., by finely grinding and mixing with a mortar and pestle, or the like) with the desiccant. Without being bound by theory, it is thought that mixing the desiccant with the thermolabile salt allows the desiccant temporarily to sequester the water of hydration from the thermolabile salt as it is heated and converted to water vapor. Advantageously, this allows the heating of the mixture of thermolabile salt and desiccant to proceed smoothly without sputtering.

The desiccant can comprise an inorganic oxide. Nonlimiting examples of suitable desiccants include silicon dioxide, aluminum oxide, phosphorous pentoxide, and zirconium oxide. Other suitable desiccant materials include clay, molecular sieves, anhydrous potassium sulfate, and anhydrous calcium sulfate. The desiccant can be any compound or mixture that temporarily absorbs or adsorbs water before and/or during the thermal decomposition of the thermolabile salt, with the proviso that the desiccant does not substantially interfere with the thermal decomposition of the thermolabile salt. Interference with the thermal decomposition includes substantially altering the thermal decomposition temperature or decomposition rate or substantially reacting with sterilant gasses produced by thermal decomposition of the thermolabile salt.

The thermolabile salt hydrate can be mixed with the desiccant in any ratio suitable to prevent liquefaction of the mixture during thermal decomposition and without substantially interfering with thermal decomposition of the thermolabile salt. In certain preferred embodiments, the mixture comprises at least enough desiccant to readily absorb or adsorb the water of hydration of the thermolabile salt hydrate. For example, in some embodiments, on a mass basis taking into account only the relative portions of thermolabile salt and desiccant, the mixture can comprise greater than 1 percent thermolabile salt, greater than 2% thermolabile salt, greater than 3% thermolabile salt, greater than 4% thermolabile salt, greater than 5% thermolabile salt, greater than 6% thermolabile salt, greater than 7% thermolabile salt, greater than 8% thermolabile salt, greater than 9% thermolabile salt, greater than 10% thermolabile salt, greater than 15% thermolabile salt, greater than 20% thermolabile salt, greater than 25% thermolabile salt, greater than 34% thermolabile salt, greater than 50% thermolabile salt, greater than 66% thermolabile salt, greater than 75% thermolabile salt, greater than 80% thermolabile salt, greater than 90% thermolabile salt, greater than 95% thermolabile salt, greater than 98% thermolabile salt, or greater than 99% thermolabile salt. In any of the above embodiments, the mixture comprising the thermolabile salt and the desiccant can comprise at least one other component that does not with the thermal decomposition of the thermolabile salt.

The thermolabile salt can be heated to a temperature sufficient to cause the decomposition of the thermolabile salt to decompose to a sterilant gas. The temperature required to decompose thermolabile salts varies according to the properties of the salt composition and information regarding the decomposition temperature for suitable thermolabile salts of nitrates and nitrites, for example, can be found in an article by K.H. Stern entitled, High Temperature Properties and Decomposition of Inorganic Salts, Part 3. Nitrates and Nitrites” (J. Phys. Chem. Ref. Data, 1972, Vol. 1, pp. 747-772), which is incorporated herein by reference in its entirety.

In an exemplary embodiment, about 1 part of a metal salt (e.g., Fe₂(NO₃)₃.9H₂O) can be uniformly admixed with about 3 parts of a desiccant (e.g., Davisil™ #1489 silica, 20-30 micron) and the mixture can be heated to a temperature sufficient to release a sterilant gas. In an exemplary embodiment, about 1 part of a metal salt (e.g., Fe₂(NO₃)₃.9H₂O) can be uniformly admixed with about 1 part of a desiccant (e.g., Davisil™ #1489 silica, 20-30 micron) and the mixture can be heated to a temperature sufficient to release a sterilant gas. In an exemplary embodiment, about 2 parts of a metal salt (e.g., Fe₂(NO₃)₃.9H₂O) can be uniformly admixed with about 1 part of a desiccant (e.g., Davisil™ #1489 silica, 20-30 micron) and the mixture can be heated to a temperature sufficient to release a sterilant gas.

Process for Generating a Sterilant Gas by the Reaction of a Metal with an Acid:

The present disclosure provides processes for generating a sterilant gas. In some embodiments, the process comprises providing an oxidizable metal and an acid that can be reduced to a sterilant gas. The process further comprises contacting the oxidizable metal and the acid under conditions suitable to cause the reduction of the acid to a sterilant gas.

In some embodiments, the oxidizable metal can comprise copper and the acid can comprise nitric acid. The metal can be contacted with the acid in a suitable container (e.g., a container that is able to maintain its integrity during exposure to the reactants and products of the reaction) at ambient temperatures to generate the following reaction:

Cu+4HNO₃→Cu(NO₃)₂+NO₂+2H₂O (4)

whereby the solid copper metal is contacted with an aqueous solution of nitric acid to produce a solution of copper nitrate in water and gaseous nitrogen dioxide. Other suitable oxidizable metals will be apparent to a person of ordinary skill in the art. Suitable acids include acids that are capable of being reduced to a sterilant gas such as, for example, nitric oxide and/or nitrogen dioxide.

It is contemplated that the reaction of an oxidizable metal with an acid capable of being reduced to a sterilant gas can be conducted in any of the sterilizers or sterilization systems disclosed herein. In these embodiments, the reaction can take place by contacting the metal and the acid in, for example, the receptacle 142 of FIG. 1 or the receptacle 242 of FIG. 2. In these embodiments, the receptacle should be constructed from materials (e.g., glass, PTFE-coated glass or metal) that are resistant to the potential corrosive effects of the reactants and/or the products. In certain preferred embodiments, the sterilizer may be modified to allow for dispensing the acid into a receptacle containing the metal (or vice versa) after the chamber is sealed. Mechanical elements to accomplish such combining processes (e.g., combining a liquid with a solid in a chamber) and, optionally, mixing processes are known in the art.

### Process for Sterilizing an Object:

The present disclosure provides a process for sterilizing an object. The process comprises contacting the object in a sterilizer with a sterilant gas generated by thermal decomposition of a thermolabile salt.

In some embodiments, the process comprises providing an object to be sterilized, a sterilizer, a source of thermal energy, and thermolabile salt. The process further comprises placing the object into the sterilizer. Preferably, the sterilizer comprises a sealable chamber, as described herein. The process further comprises heating the thermolabile salt to a temperature sufficient to cause the salt to decompose to generate a sterilant gas. In some embodiments, the thermolabile salt may be disposed in a sachet. The thermolabile salt can be heated in a gas-generating module, as described herein. The process further comprises receiving the sterilant gas in the sterilizer. In some embodiments, the gas-generating module can be disposed in the sterilizer whereby, upon heating the thermolabile salt in the gas-generating module, the sterilant gas is released into the sterilizer. In some embodiments, the gas generating module can be in fluid communication with the sterilizer whereby, upon heating the thermolabile salt in the gas-generating module, the sterilant gas is transferred into the sterilizer. In some embodiments, the fluid communication can be selective fluid communication (e.g., regulated by one or more valves). In some embodiments, the sterilant gas can be transferred into the sterilizer by positive and/or negative pressure. The process further comprises contacting the object with the sterilant gas in the sterilizer for a period of time.

In any of the above processes for sterilizing an object, the thermolabile salt can comprise an inorganic salt. In any of the above processes, the thermolabile salt can comprise a nitrate salt, a nitrite salt, a chlorate salt, a perchlorate salt, or mixtures thereof. In some embodiments, the thermolabile salt decomposes to produce an oxide of nitrogen (e.g., nitric oxide (NO), nitrogen dioxide (NO₂), dinitrogen trioxide (N₂O₃), dinitrogen tetroxide (N₂O₄), dinitrogen pentoxide (N₂O₅) and/or nitrous oxide (N₂O). In any of the above processes, the thermolabile salt can comprise a salt hydrate. In some embodiments, the thermolabile salt can comprise Ba(NO₃)₂, AgNO₃, Fe(NO₃)₃.9H₂O, Cu(NO₃)₂.2.5H₂O, Ca(NO₃)₂.4H₂O, Mn(NO₃)₂.4H₂O, Co(NO₃)₂.6H₂O, and Zn(NO₃)₂.xH₂O.

In any of the above embodiments, generating a sterilant gas can comprise generating the gas in the presence of an oxygen source (e.g., air, oxygen, or a mixed gas comprising oxygen). As described herein, certain oxides of nitrogen can react with oxygen to produce additional oxides of nitrogen. In some embodiments, as the thermolabile salt is heated, it may be heated in the presence of oxygen. Alternatively or additionally, after the thermolabile salt has decomposed, at least one gaseous product of the decomposition process can be contacted with oxygen.

In any of the above embodiments the object in the sterilizer can be contacted with water vapor (e.g., humidified air) before, during, or after the object is contacted with the sterilant gas. In some embodiments, the water vapor comprises about 20% to about 99% relative humidity. In some embodiments, the water vapor comprises about 30% to about 90% relative humidity. In some embodiments, the water vapor comprises about 40% to about 80% relative humidity. The water vapor can be provided by a humidifier, vaporizer, or a steam line, for example.

### Containers for Gaseous Sterilant-Generating Salts:

Methods of the present disclosure include providing a thermolabile salt capable of decomposing to produce a sterilant gas. Preferably, the salt is provides in the form of a solid material. In some embodiments, the thermolabile salts can be provided as part of a mixture (e.g., as a mixture of two or more distinct thermolabile salts, as a mixture of one or more thermolabile salts and a desiccant). In some embodiments, the thermolabile salt, or mixtures thereof, can be added directly to a gas-generating module to allow for the thermal decomposition of the salt.

In some embodiments, the thermolabile salt, or mixtures thereof, can be provided in a container (e.g., a cartridge or a sachet) so that the salt can be handled by a technician with greater convenience. The container may contain an amount of thermolabile salt sufficient for a single sterilization process. The container may contain an amount of thermolabile salt sufficient for two or more sterilization processes. The container may comprise openings, to release the sterilant gas. The container may comprise one or more frangible seals, which can be opened before or during use, as described above.

The container can be made of any suitable material that is adapted for heating the thermolabile salt to a temperature at which it decomposes. Suitable materials are sufficiently nonporous and structurally stable to hold the thermolabile salt during handling by the technician. Furthermore, the materials allow for the transfer of thermal energy from a thermal energy source to the thermolabile salt. The container can be formed into various shapes and/or sizes. In some embodiments, the container is dimensioned to fit easily into the receptacle of a gas-generating module. In some embodiments, the container may be constructed of materials (e.g., certain metals, ceramics) that are resistant to the elevated temperatures to which the thermolabile salts are heated for decomposition. In some embodiments, the containers may be constructed from materials that can degrade at the elevated temperatures to which the thermolabile salts are heated for decomposition.

The invention will be further illustrated by reference to the following non-limiting Examples. All parts and percentages are expressed as parts by weight unless otherwise indicated.

### EXAMPLE S

### Examples 1-6

About 200 milligrams of Iron(III)nitrate nonahydrate was placed in a test tube and heated with a Bunsen burner flame. Upon initial heating the salt liquefied. The liquefied substance sputtered and a gas was emitted that appeared to be water vapor. After further heating and most of the water vapor came off, a reddish brown gas was emitted, indicating the formation of nitrogen dioxide. The procedure was repeated with each hydrated metal nitrate salt listed in Table 1. Each of the salts liquefied, emitted water vapor and sputtered, and then emitted nitrogen dioxide.

TABLE 1
Hydrated Metal Salts*

| Ex | Metal salt | Supplier |
|---|---|---|
| 1 | Iron(III)nitrate nonahydrate—Fe(NO₃)₃•9H2O | Alfa Aesar, Ward |
| | | Hill, MA |
| 2 | Cupric nitrate hydrate—Cu(NO₃)₂•2.5H2O | J. T. Baker, |
| | | Phillipsburg, NJ |
| 3 | Calcium nitrate tetrahydrate—Ca(NO₃)₂•4H2O | VWR West |
| | | Chester, PA |
| 4 | Manganese(II)nitrate | Alfa Aesar, Ward |
| | tetrahydrate—Mn(NO₃)₂•4H2O | Hill, MA |
| 5 | Cobalt(II) nitrate hexahydrate—Co(NO₃)₂•6H2O | Alfa Aesar, Ward |
| | | Hill, MA |
| 6 | Zinc nitrate hydrate—Zn(NO₃)₂•xH2O | Alfa Aesar, Ward |
| | | Hill, MA |

*All metal salts were at least 98% pure on a metal basis.

### Examples 7-9

Non-hydrated metal salts, obtained from Alfa Aesar, Ward Hill, Mass., were heated to form nitrogen dioxide using the procedure of Example 1. The salts were: Example 7—barium nitrate, Example 8—potassium nitrate and Example 9—silver nitrate. No emission of water vapor was observed for any of the salts. The barium and silver salts melted and decomposed to yield nitrogen dioxide with no sputtering. No nitrogen dioxide evolved from the potassium nitrate at the temperature to which it was heated in the test tube using the Bunsen burner flame.

### Examples 10-11

Iron(III)nitrate nonahydrate was mixed with silica (Davisil™ #1489 silica, 20-30 micron, available from Alltech Associates, Deerfield, Ill.) at a weight ratio of about 1 part metal nitrate salt to about 3 parts silica using a mortar and pestle to form a substantially uniform mixture. About 200 milligrams of this mixture was placed in a test tube and heated according to the procedure of Example 1. The same procedure was repeated with cupric nitrate hydrate for Example 11. Each mixture of metal nitrate salt and silica emitted nitrogen dioxide with no sputtering.

### Examples 12-14

Iron(III)nitrate nonahydrate was mixed with silica (as described in Example 10) at the weight ratios shown in Table 2. About 200 milligrams of this mixture was placed in a test tube and heated according to the procedure of Example 1. None of the mixtures liquefied during the heating process. Each mixture of metal nitrate salt and silica remained powdered when heated and each mixture emitted nitrogen dioxide with no sputtering.

TABLE 2
Ratio of salt hydrate to silica.

| | Iron(III)nitrate nonahydrate | Silica |
|---|---|---|
| Example | (weight %) | (weight %) |
| 12 | 25 | 75 |
| 13 | 50 | 50 |
| 14 | 66 | 34 |

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

1. A process for producing a sterilant gas, comprising:
providing a source of thermal energy and a mixture comprising a desiccant and a thermolabile salt; and
heating the mixture to a temperature sufficient to cause decomposition of the salt to at least one sterilant gas.

2. The process of claim 1, wherein the thermolabile salt comprises a salt hydrate.

3. The process of claim 1, wherein the thermolabile salt comprises a nitrite salt, nitrate salt, a chlorate salt or a perchlorate salt.

4. The process of claim 3, wherein the nitrate salt comprises Ba(NO₃)₂, AgNO₃, Fe(NO₃)₃.9H₂O, Cu(NO₃)₂.2.5H₂O, Ca(NO₃)₂.4H₂O, Mn(NO₃)₂.4H₂O, Co(NO₃)₂.6H₂O, or Zn(NO₃)₂.xH₂O.)

5. The process of claim 1, wherein heating the thermolabile salt comprises heating the salt in the presence of oxygen.

6. The process of claim 1, wherein the desiccant does not substantially interfere with the thermal decomposition of the thermolabile salt.

7. The process of claim 1, wherein the desiccant comprises a molecular sieve, clay, anhydrous potassium sulfate, anhydrous calcium sulfate, an inorganic oxide, or mixtures thereof.

8. The process of claim 7, wherein the inorganic oxide is selected from the group consisting of silicon dioxide, aluminum oxide, and zirconium oxide.

9. The process of claim 1 wherein the salt is disposed in a package adapted for heating the salt to a temperature sufficient to cause decomposition of the salt to a sterilant gas.

10-26. (canceled)

27. A composition for generating a sterilizing gas, comprising a desiccant and a thermolabile salt hydrate, wherein the thermolabile salt is capable of decomposing at an elevated temperature to generate a sterilant gas and wherein, on a mass basis, the composition comprises greater than one part thermolabile salt per nine parts desiccant.

28. The composition of claim 27, wherein the thermolabile salt hydrate comprises a nitrate salt, a nitrite salt, a sulfate salt, a sulfite salt, a chlorate salt, a perchlorate salt, or mixtures thereof.

29. The composition of claim 28, wherein the salt hydrate is selected from the group consisting of Fe(NO₃)₃.9H₂O, Cu(NO₃)₂.2.5H₂O, Ca(NO₃)₂.4H₂O, Mn(NO₃)₂.4H₂O, Co(NO₃)₂.6H₂O, and Zn(NO₃)₂.xH₂O.

30. The composition of claim 27, wherein the desiccant comprises a molecular sieve, clay, anhydrous potassium sulfate, anhydrous calcium sulfate, an inorganic oxide, or mixtures thereof.

31. The composition of claim 30, wherein the inorganic oxide is selected from the group consisting of silicon dioxide, aluminum oxide, and zirconium oxide.

32. A system for sterilizing an object, comprising a sterilization chamber, a heat source, and a thermolabile salt, wherein the thermolabile salt is capable of decomposing at an elevated temperature to generate a sterilant gas.

33. The system of claim 32, wherein the sterilization chamber is sealable.

34. The system or of claim 32, wherein the thermolabile salt comprises a nitrate salt, a nitrite salt, a chlorate salt, a perchlorate salt, or mixtures thereof.

35. The system of claim 32, wherein the thermolabile salt comprises a salt hydrate.

36. The system of claim 35, wherein the salt hydrate is selected from the group consisting of Fe(NO₃)₃.9H₂O, Cu(NO₃)₂.2.5H₂O, Ca(NO₃)₂.4H₂O, Mn(NO₃)₂.4H₂O, Co(NO₃)₂.6H₂O, and Zn(NO₃)₂.xH₂O.

37. The system of claim 32, wherein the thermolabile salt is admixed with a desiccant.

38. The system of claim 32, wherein the desiccant comprises a molecular sieve, clay, anhydrous potassium sulfate, anhydrous calcium sulfate, an inorganic oxide, or mixtures thereof.

39. The system of claim 38, wherein the inorganic oxide is selected from the group consisting of silicon dioxide, aluminum oxide, and zirconium oxide.

40. The system of claim 32, further comprising a gas-generating chamber.

41. The system of claim 32, further comprising a source of oxygen.

42. The system of claim 41, wherein the source of oxygen comprises air.

43. The system of claim 32, further comprising a source of moisture vapor.

44. The system of claim 32, further comprising a vacuum source.

45. The system of claim 32, wherein the thermolabile is disposed in a package adapted for heating the salt to a temperature sufficient to cause decomposition of the salt.

46. The system of claim 45, wherein the package contains an amount of thermolabile salt sufficient for a single sterilization process.

47. The system of claim 32, further comprising a gas-scrubbing component.

48-52. (canceled)
